(19) United States
(12) Patent Application Publication
(10) Pub. No.: US 2006/0147814 A1
(43) Pub. Date: Jul. 6, 2006

(54) Methods for repairing an alternating phase-shift mask

(76) Inventor: Ted Liang, Sunnyvale, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/028,818

(22) Filed: Jan. 3, 2005

(51) Int. Cl. G21G 5/00; G03C 5/00; A61N 5/00; G03F 1/00

(52) U.S. Cl. 430/5; 250/492.3; 430/322; 430/323; 430/324

(57) ABSTRACT

Methods to repair an APSM mask having undercut etch are described. An absorbing layer over a defect on the plate and a first portion of a defect on the plate are removed using a tip of an atomic force microscope. A second portion of the defect is removed using an e-beam induced etching, which includes introducing a first gas over a second portion of the defect to form a first chemistry to etch the defect, and dwelling the e-beam. The absorbing layer having an overhung structure is reconstructed on the plate using an e-beam induced deposition. A second gas is introduced over the plate to form a second chemistry to form an opaque material on the plate. The e-beam is dwelled for a predetermined time to induce forming the opaque material on the plate. For an embodiment, a profile of the defect is measured to control etching.

### FIELD

Embodiments of the invention relate generally to the field of mask manufacturing, and more specifically, to methods of mask repairing.

### BACKGROUND

Phase-Shift Mask (“PSM”) technology has been pioneered in recent years to extend the limits of optical lithography. Typically, a photomask is composed of quartz and chrome features. Light passes through the clear quartz areas and is blocked by the opaque chrome areas. Where the light hits the wafer, the photoresist is exposed, and those areas are later removed in the develop process, leaving the unexposed areas as features on the wafer. As feature sizes and pitches shrink, the resolution of the projection optics begins to limit the quality of the resist image. There is a significant intensity of the light, which is proportional to the square of the energy even below the opaque chrome areas, due to the very close proximity of the neighboring clear quartz areas. The light below opaque chrome areas affects the quality of the resist profiles, which are ideally vertical. Therefore phase-shift techniques are designed to “sharpen” the intensity profile, and thus the resist profile, which allows smaller features to be printed.

PSM technology includes an Alternating Phase-Shift (“APS”) mask technology, which typically employs alternating areas of an absorbing layer of chrome and a 180 degree phase shifted quartz plate to form features on the wafer. An APS mask enhances the optical resolution, a contrast of the projected image, and increases the depth of focus of a lithography process for wafer printing.

FIG. 1 illustrates a system 100, wherein the light 101 passes through an APS mask 102 and reaches a wafer 103 covered by a photoresist 104. The APS mask 102 has areas 105 of an absorbing layer of chrome on a quartz plate 106. As shown in FIG. 1, light 101 passes through the quartz areas 107 and 108 and is blocked by the areas 105 of the absorbing layer. The light 101 passed through the quartz areas 107 and 108 reaches areas 110 of a photoresist covering the wafer 103. As shown in FIG. 1, the areas 110 of the photoresist exposed by the light 101 are later removed in a photoresist developing process, leaving the unexposed areas 104 of the photoresist as features on the wafer. As shown in FIG. 1, the thickness of the quartz plate 106 in the area 107 corresponds to 0 degree phase of the light 101, and the thickness of the quartz plate 106 in the area 108 corresponds to 180 degree phase of the light 101. As the phase of the light 101 passing through the areas 107 and 108 changes respectively from 0 degree to 180 degree, it passes through zero. The intensity of the light, which is proportional to the square of the amplitude of the light, also passes zero, making a dark and sharp line on the wafer. The intensity of the light 101 transmitted through areas 107 and 108 of the APS mask 102 is, however, imbalanced, for example, because of scattering of the light 101 by sidewalls, as shown in FIG. 1A. The intensity imbalance in the APS mask can result in errors in a resolution, errors in a phase, and errors in a placement on the wafer.

FIG. 1B illustrates an APS mask 121, wherein the quartz plate 126 above the absorbing layer 125 of chrome is etched creating an undercut 124 to prevent the intensity imbalance of the light 122 in the APS mask 121. Typically, isotropic wet etching is used to form the undercut 124. As shown in FIG. 1B, the undercut 124 creates an overhung structure in the absorbing layer 125. The overhung structure may lead to peeling off the absorbing layer 125, because there may be not enough quartz to support the absorbing layer 125. In particular, the absorbing layer 125 may be peeled off if the undercut 124 is larger than a width of the quartz plate 126 between the 0 degree and 180 degree phase trenches.

FIGS. 2A to 2C illustrate various types of defects on an APS mask 200. FIG. 2A illustrates the APS mask 200 having an absorbing layer 202 of chrome on a plate 201 of quartz. FIG. 2A illustrates a defect 203, which includes a bump of quartz adjacent to a sidewall of a trench 204. FIG. 2B illustrates the APS mask 200 with a defect 205, which includes a bump of quartz on a bottom of the trench 204. FIG. 2C illustrates the APS mask 200 having a missing piece 206 of the absorbing layer 202 of chrome on the plate 201 of quartz.

Currently there is no technique to repair the defects 203 and 205 illustrated in FIGS. 2A and 2B, which maintains the undercut and overhung structures in the APS mask. Typically, a focused ion beam (“FIB”) having Ga ions is used to remove the defects 203 and 205. However, FIB results in Ga staining 301 on the sidewalls and on the bottom of the trench 204 in the plate 201 of the quartz, as shown in FIG. 3A. Ga staining causes the transmission loss and requires post repair treatment of the ASP mask. In addition, as shown in FIG. 3A, FIB removes the overhung absorbing layer that leads to the light intensity imbalance in the APS mask discussed above.

Another method to remove the defect 205 of FIG. 2B uses an Atomic Force Microscope (“AFM”) tip 302 to mechanically remove the defect 205 on the bottom of the trench 204. As illustrated in FIG. 3B, the AFM tip 302 may cut the defect 205 only when the size of the trench 204 in the plate 201 is substantially larger than the size of the AFM tip 302. In addition, because the AFM tip 302 has a tapered shape, it can either damage the sidewall of the trench 204, or will not be able to reach into the trench 204 and completely remove the defect 205, as shown in FIG. 3B. Further, the AFM tip, similar to the FIB, removes overhung absorbing layer abolishing the undercut, as shown in FIG. 3B. All of that causes the light intensity imbalance in the APS mask, which is discussed above.

### BRIEF DESCRIPTION OF THE DRAWINGS

The present invention is illustrated by way of example and not limitation in the figures of the accompanying drawings, in which like references indicate similar elements, in which:

FIG. 1 illustrates a typical prior art system, wherein the light passes through an APS mask and reaches a wafer;

FIGS. 2A to 2C illustrate various prior art types of defects on an APS mask;

FIGS. 3A and 3B illustrate prior art methods to remove defects on an APS mask;

FIG. 4A is a side view of an APS mask, wherein a tip is used to mechanically remove an absorber over a defect according to one embodiment of the invention;

FIG. 4B is a side view of an APS mask, wherein an e-beam is used to remove an absorber over a defect according to another embodiment of the invention;

FIG. 4C is a view similar to FIGS. 4A and 4B, wherein an e-beam is used to remove the defect according to one embodiment of the invention;

FIG. 4D is a view similar to FIG. 4C, wherein an e-beam is used to reconstruct an absorber having an overhung structure according to one embodiment of the invention;

FIG. 5 illustrates an e-beam induced etching of a defect on a plate of an APS mask according to one embodiment of the invention;

FIG. 6 is a block diagram of the AFM-based system to measure a three dimensional profile of a defect on a plate of an APS mask according to one embodiment of the invention;

FIG. 7 illustrates depositing of an absorbing layer having an overhung structure on a plate of an APS mask using an e-beam induced deposition according to one embodiment of the invention;

FIG. 8 is a side view of an APS mask, wherein an e-beam is used to reconstruct a missing absorber having overhung structures according to another embodiment of the invention.

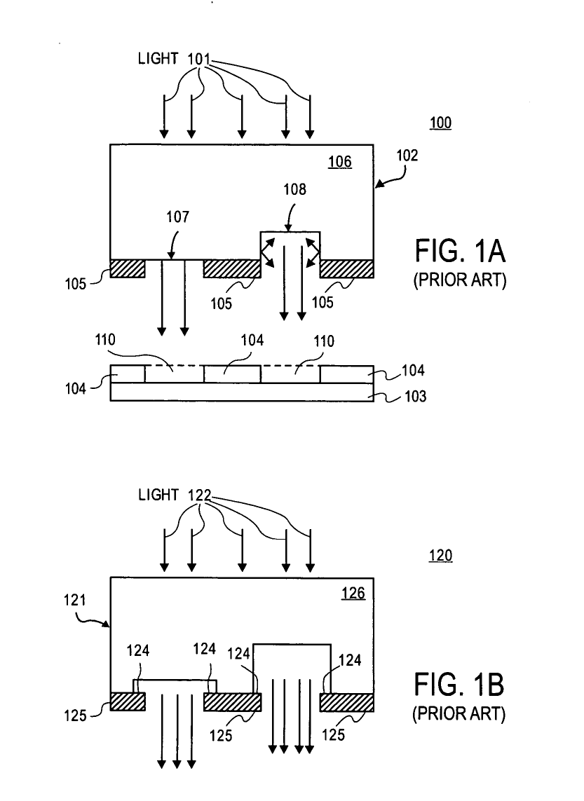

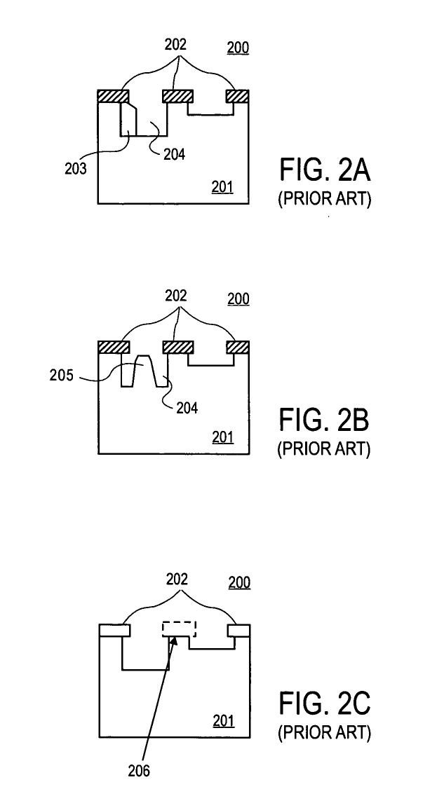

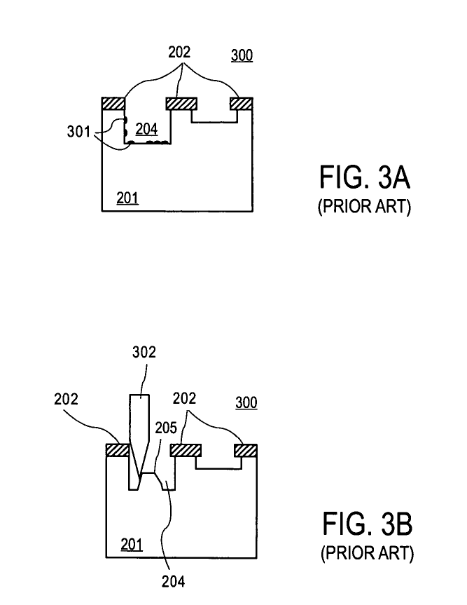

### DETAILED DESCRIPTION

In the following description, numerous specific details, such as specific materials, chemistries, dimensions of the elements, etc. are set forth in order to provide thorough understanding of one or more of the embodiments of the present invention. It will be apparent, however, to one of ordinary skill in the art that the one or more embodiments of the present invention may be practiced without these specific details. In other instances, semiconductor fabrication processes, techniques, materials, equipment, etc., have not been described in great details to avoid unnecessarily obscuring of this description. Those of ordinary skill in the art, with the included description, will be able to implement appropriate functionality without undue experimentation.

While certain exemplary embodiments of the invention are described and shown in the accompanying drawings, it is to be understood that such embodiments are merely illustrative and not restrictive of the current invention, and that this invention is not restricted to the specific constructions and arrangements shown and described because modifications may occur to those ordinarily skilled in the art.

Reference throughout the specification to “one embodiment”, “another embodiment”, or “an embodiment” means that a particular feature, structure, or characteristic described in connection with the embodiment is included in at least one embodiment of the present invention. Thus, the appearance of the phrases “for one embodiment” or “for an embodiment” in various places throughout the specification are not necessarily all referring to the same embodiment. Furthermore, the particular features, structures, or characteristics may be combined in any suitable manner in one or more embodiments.

Moreover, inventive aspects lie in less than all the features of a single disclosed embodiment. Thus, the claims following the Detailed Description are hereby expressly incorporated into this Detailed Description, with each claim standing on its own as a separate embodiment of this invention. While the invention has been described in terms of several embodiments, those skilled in the art will recognize that the invention is not limited to the embodiments described, but can be practiced with modification and alteration within the spirit and scope of the appended claims. The description is thus to be regarded as illustrative rather than limiting.

Methods to repair an alternating phase-shift (“APS”) mask that maintain the phase and intensity balance of the light are described herein. The methods include repairing defects on a mask having an etch undercut structures for balancing light intensity. In particular, methods include removing defects in the undercut regions of the plate supporting an absorbing layer (“absorber”), and reconstructing the absorbing layer having an overhung structure on the plate of the mask. In an embodiment, first, an absorbing layer over a defect is removed using an Atomic Force Microscope (“AFM”) tip, an electron beam (“e-beam”), or a combination thereof. The defect is removed from the plate using an e-beam induced etching with a first chemistry. Further, an absorbing layer having an overhung structure is reconstructed on the plate by redepositing an opaque material on the plate using an e-beam induced deposition with a second chemistry. For one embodiment, to repair a defect having a missing absorber on the plate, the e-beam induced deposition of the opaque material is used. For one embodiment, a three-dimensional (“3D”) profile of the defect on the plate of the mask is generated to control removing of the defect. The methods described herein do not damage the mask, do not cause a transmission loss in the mask, and therefore do not require a post repair treatment of the mask. The methods described herein provide substantially high spatial resolution that allows repairing the masks having substantially small dimensions and substantially small defects.

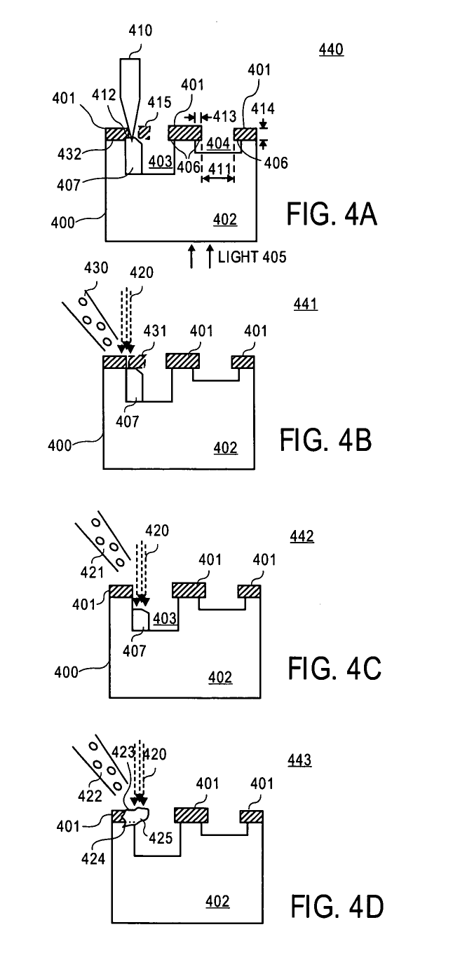

FIG. 4A is a side view 440 of an APS mask 400, wherein a tip 410 is used to mechanically remove an absorber 401 over a defect 407 adjacent to a sidewall of a trench 403 in a plate 402 according to one embodiment of the invention. As shown in FIG. 4A, the APS mask 400 has a trench 403 and a trench 404 etched into the plate 402 adjacent to each other, as shown in FIG. 4A. Each of the trenches 403 and 404 in the plate 402 has undercut (“overhung”) structures 406, which include a portion of the absorber 401 above the trench, which is not supported by the plate 402, as shown in FIG. 4A. The overhung structures 406 are formed to balance the intensity of the light 405, by, for example, reducing scattering of the light 405 transmitting through the plate 402 from the sidewalls of the trenches 403 and 404. For one embodiment, the plate 402 of the APS mask 400 may be any material that is transparent for the light 405 and the absorber 401 on the plate 402 may be any material blocks the light 405. More specifically, a material for the plate 402 of the APS mask 400 may be quartz, glass, or any combination thereof. The absorber 401 on the plate 402 may be chrome, tantalum nitride, molybdenum silicide, or any combination thereof. For an embodiment, the light 405, which is transparent for the plate 402 and is opaque for the absorber 401, may be extreme ultraviolet, ultraviolet, x-ray, or any combination thereof. For one embodiment, the width 411 of each of the openings in the absorber 401 on the plate 402 is in the approximate range of 100 nm to 500 nm. A depth of the trench 403 relates to a depth of the trench 404 such that a phase of light 405 transmitted through the plate 402 and trench 403 is shifted by 180 degree relative to the phase of the light 405 transmitted through the plate 402 and trench 404. Generally, a change of the phase (“Δθ”) for the light 405 transmitted through the plate 402 depends on the index of refraction of the material of the plate 402, a depth of the etched trench in the plate 402, and a wavelength of the light 405 according to a formula below:
Δθ˜2π(n−1)d/λ,
where λ is the wavelength of the light, n-is the index of refraction of the material of the plate 402, and d is a depth of an etched trench in the plate 402. The index of refraction n depends on the material of the plate 402 and on the wavelength of the light 405. For example, for the light 405 having a wavelength of 193 nm from the stepper, the index of refraction of quartz is about 1.55. For an embodiment, a respective depth for each of the trenches 403 and 404 may be calculated according to the above formula. Each of the undercut structures 406 has a length 413 measured from the sidewall of the trench to the edge of the respective absorber. For one embodiment, the length 413 of each of the undercut structures 406 is in the approximate range of 20 nm to 150 nm. More specifically, the length 413 of each of the undercut structures 406 may be between 30 nm and 60 nm. The trenches 403 and 404 and undercut structures 406 of the APS mask 400 may be formed by wet etch, dry etch, or a combination thereof using one of techniques known to one of ordinary skill in the art of mask fabrication. For an embodiment, a thickness 414 of the absorber 401 of chrome on the plate 402 of quartz is in the approximate range of 50 nm to 150 nm. For an embodiment, the defect 403 on the plate 402 of quartz under the absorber 401 of chrome may be a quartz bump having a size in the approximate range of 20 nm to few hundreds of nanometers, depending on the mask fabrication process and lithographic wafer printing requirements, for example, from 20 nm to 900 nm.

Further, the absorber 401 is mechanically removed by cutting through the absorber 401 down to the defect 407 using the tip 410. After cutting through the absorber 401, the tip 410 may continue to cut the defect 407 until a sidewall of the tip 410 touches a sidewall of the trench 403. For one embodiment, the tip 410 to cut the absorber 401 of chrome over the defect 407 on the plate 402 of quartz is a tip of an Atomic Force Microscope (“AFM”). AFM may be used as a scanning tool to control the cutting thickness based on the height information provided by the tip and control electronics of the AFM. Scanning speed of about 1 micron per second may be used to minimize wearing of the tip 410. Each pass (“feed”) of the AFM tip may provide about 1 nm cut into the absorber 401. For one embodiment, to remove the absorber 401 of chrome having a thickness of in the approximate range of 50 nm to 100 nm over the defect 407, the AFM tip may have about 100 to about 150 passes over the absorber 401. For an embodiment, a portion 412 of the absorber 401 may be removed from a portion of a top 432 of the plate 402 to provide sufficient space for reconstructing the absorber later on in the process. More specifically, the portion 412 supported by the plate may be in the approximate range of 10 nm to 50 nm.

After removing the absorber 401, the tip 410 may continue to cut the defect 407 into a predetermined depth to ensure that the absorber 401 over the defect 407 is completely removed. For one embodiment, the tip 410 may cut the defect 407 of quartz to the predetermined depth, which is in the approximate range of 3 nm to 15 nm. For one embodiment, the tip 410 may be the tip of 650 nm and 1300 nm AFM machining equipment manufactured by RAVE, LLC., located in Delray Beach, Fla., may be used to mechanically cut the absorber 401 and a portion of the defect 407.

Next, a debris 415 that may result from the mechanical cutting of the absorber 401 and a portion of the defect 407 is removed. The debris 415 may be removed between passes of AFM tip and after completing cutting the absorber 401 and a portion of the defect 407. Removing of the debris 415 may be performed by first loosening the debris 415 from a surface of the APS mask 400, and then cleaning the debris from the APS mask 400. For an embodiment, removing the debris 415 from the surface of the APS mask 400 may be performed using a flow of gas. For one embodiment, the carbon dioxide gas in a critical state that includes dry ice particles is used to remove the debris resulted from cutting of the absorber 401 and the portion of the defect 407.

FIG. 4B is a side view 441 of the APS mask 400, wherein the e-beam 420 is used to remove an absorber 401 over a defect 407 on the plate 402 according to another embodiment of the invention. Removing the absorber 401 over the defect 407 is performed by etching the absorber 401, wherein etching is induced by an e-beam 420, as shown in FIG. 4B. A precursor gas 430 is dispensed near the e-beam 420. The e-beam 420 is focused on a portion 431 of the absorber 401 to be etched, as shown in FIG. 4B. The e-beam 420 induces a chemical reaction to etch the portion 431 of the absorber 401. Etching is enabled by a chemical reaction between the precursor gas 430 and a material of the absorber 401 resulting in volatile products. For one embodiment, the precursor gas 430 to etch the portion 431 of the absorber 401 includes oxygen. For another embodiment, the precursor gas to etch the portion 431 includes chlorine. For one embodiment, the precursor gas 430 to etch the portion 431 is oxygen, chlorine, a fluorine containing gas, for example, XeF₂, or any combination thereof, wherein the absorber 401 is tantalum nitride, chrome, molybdenum silicide, or any combination thereof.

The choice of etching the absorber 401 with the e-beam 420 as opposed to mechanical cutting the absorber 401 with the tip 410 depends on removal selectivity of the material of the absorber 401 relative to the material of the plate 402. Higher removal selectivity for material of the absorber 401 relative to the material of the plate 402 means that the absorber 401 is removed substantially faster than the material of the plate 402, such that the removal process is substantially slowed down at the interface between the plate 402 and the absorber 401. The use of the e-beam as opposed to the AFM tip is also determined by the lowest damage to the substrate while removing the absorber. For one embodiment, the absorber 401 of tantalum nitride over the defect 407 on the plate 402 of quartz is removed using the e-beam 420. For another embodiment, the absorber 401 of chrome over the defect 407 on the plate of quartz is removed using the mechanical cutting with the tip 410.

For an embodiment, prior to using an e-beam, hydrocarbons are removed from the surface of the APS mask 400. Hydrocarbons are removed, because hydrocarbons may be activated by the e-beam later on in the process producing carbon molecules that may prevent etching of the defect 407. Depending on the amount of hydrocarbons, wet cleaning using an acid, dry cleaning using an ozone, or a combination thereof may be used to remove hydrocarbons from the surface of the APS mask 400. For one embodiment, the surface of the APS mask 400 may be cleaned with 96% sulfuric acid for about 10 minutes and then cleaned with the ozone for about 4 to 5 minutes. Techniques to clean a surface from hydrocarbons are well known to one of ordinary skill in the art of mask fabrication. Next, the defect 407 on the plate 402 of the APS mask 400 is removed by e-beam induced etching.

FIG. 4C is a side view 442 of the APS mask 400, wherein an e-beam 420 is used to remove the defect 407 adjacent to a sidewall of a trench 403 in the plate 402 according to one embodiment of the invention. A precursor gas 421 is dispensed near the e-beam 420 over the defect 407. The e-beam 420 is focused on a portion of the defect 407 to be etched, as shown in FIG. 4C. The e-beam 420 induces a chemical reaction to etch the defect 407. Etching is enabled by a chemical reaction between the precursor gas 421 and a material of the defect 407 resulting in volatile products. For one embodiment, a second portion of the defect 407 is removed using the e-beam 420 after a first portion of the defect 407 is removed using the tip 410, as described above with respect to FIG. 4A. For another embodiment, the defect 407 is removed using the e-beam 420 after the absorber 401 above the defect 407 is removed using the e-beam 420 with the gas 430, as described above with respect to FIG. 4B.

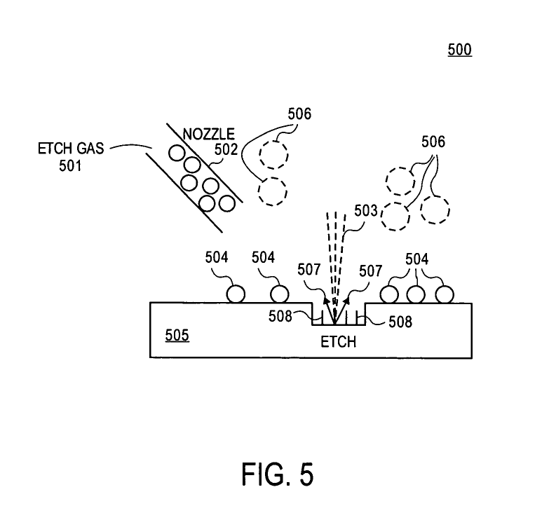

FIG. 5 illustrates a schematics 500 of an e-beam induced etching of a defect according to one embodiment of the invention. A precursor gas 501 is introduced through a nozzle 502 near a focused e-beam 503. For an embodiment, a pressure of the precursor gas 421 in the nozzle 502 is controlled to maintain the functionality of the e-beam 503. The precursor gas molecules 504 are adsorbed at a surface of a defect 505, and a chemical reaction is induced by the e-beam 503. As shown in FIG. 5, primary electrons of the e-beam 503 that hit the surface of the defect 505 cause secondary electron emission 507. Secondary electron emission 507 produces ions and radicals 508 from the molecules 504 adsorbed at the surface of the defect 505. Ions and radicals 508 produced by the secondary electron emission 507 form a first chemistry to etch into the surface of the defect 505 by a chemical reaction, which forms volatile products 506 including atoms and molecules of the material of the defect 505.

Referring back to FIG. 4C, for one embodiment, to etch the defect 407 of the quartz bump, the precursor gas 421 that includes fluorine (“F”), for example, xenon difluoride (“XeF₂”), is used. A voltage of the e-beam 420 is selected to limit charging of the etching surface. For one embodiment, a voltage of the e-beam 420 to etch the defect 407 is in the approximate range of 0.8 kilovolts (“kV”) to 1.5 kV to provide the total electron yield from the surface of the defect 407 around 1 and the diameter of the e-beam is in the approximate range of 2 nm to 6 nm. More specifically, the diameter of the e-beam 430 to remove defect 407 of quartz bump on the plate 402 of the APS mask 400 is about 5 nm, the voltage of the e-beam is about 1 kV. To remove the defect 407, the e-beam 420 is dwelled for a predetermined time over a portion of the defect 407 to be etched and then moved by a predetermined step along a line to a next point over the defect 407 performing a raster scan or a serpentine scan, which results in the e-beam scanning over the whole defect. The raster scan and serpentine scan techniques for the e-beam are well known to one of ordinary skill in the art of mask manufacturing. One frame (“loop”) corresponds to a single scan (“pass”) of the e-beam 420 over the entire defect. Next, dwelling of the e-beam 420 over the portion of the defect and moving the e-beam 420 for the predetermined step are continuously repeated until the whole defect 407 is etched away from the plate 402 of the APS mask 400. For an embodiment, time between each of the line scans (“line refresh time”) and time between each of the frame scans (“frame refresh time”) is long enough to allow the molecules of the precursor gas 421 to adsorb on the surface of the defect 407. More specifically, to etch the defect 407 of quartz having the size in the approximate range of 20 nm to 100 nm, each of the line refresh time and the frame refresh time is longer than 100 microseconds (“psec”). Typically, longer the loop, less frame refresh time is needed. For one embodiment, the predetermined step to move the e-beam 420 to the next point over the defect 407 is such that neighboring pixels defining the predetermined step, do not overlap. More specifically, the predetermined step to move the e-beam 420 to the next point over the defect 407 is in the approximate range of 2 nm to 10 nm. Size of each pixel corresponds to the portion of the defect 407 etched by the e-beam 420 during dwelling time. Parameters to control scanning of the e-beam 420 over the defect 407 depend on the size of the defect. For an embodiment, before performing e-beam induced etching of the defect 407, a three-dimensional profile of the defect 407 may be generated. For one embodiment, the profile of the defect 407 may be generated using an AFM-based system.

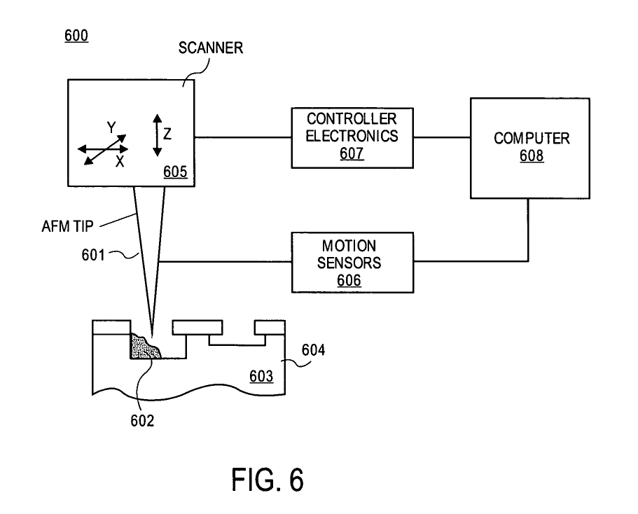

FIG. 6 is a block diagram 600 of the AFM-based system to measure a three dimensional profile of a defect on a plate of the mask according to one embodiment of the invention. As shown in FIG. 6, a tip 601 of AFM is scanned over the surface of a defect 602 on a plate 603 of a mask 604 to map the surface of the defect 602. For one embodiment, a scanner 605 moves the tip 601 over the surface of the defect 602 in two horizontal X, Y, and a vertical Z directions, as shown in FIG. 6. For another embodiment, the tip 601 may be stationary and the mask 604 may be scanned under it. A motion sensor 606, shown in FIG. 6, is coupled to the tip 601. The motion sensor 606 senses the force between the tip 601 and the surface of the defect 602, which is typically in the range of interatomic forces in solids. The motion sensor 606 provides a correction signal to the scanner 605 to keep the force constant. A controller electronics 607 provides interfacing between a computer 608, the scanner 605, and the motion sensor 606, as shown in FIG. 6. The controller electronics 607 supplies voltages that control the scanner 605, accepts the signal from the motion sensor 606, and includes the feedback control system for keeping the force between the surface of the defect 602 and the tip 601 constant. As shown in FIG. 6, a computer 608 is coupled to the controller electronics 607 and the motion sensor 606 to drive the system 600, to process, display, and analyze data to produce a three-dimensional profile of the defect 602 on the plate 603 of the mask 604.

For another embodiment, an X-Y image of the defect 407 may be obtained using electron or optical microscopy and the height of the defect may be obtained using the AFM microscope.

Referring back to FIG. 4C, parameters of the e-beam 420 to etch the defect 407 may be defined using a profile of the defect, for example, the three-dimensional profile produced by an AFM-based technique, which is described above with respect to FIG. 6. The dose of the electrons provided by the e-beam 420 to induce etching a portion of the defect 407 is defined as a product of the accumulated dwelling time of the e-beam 420 and a current of the e-beam 420. Smaller current may be compensated by longer dwelling time to produce the same dose of the electrons in the e-beam. Typically, smaller current of the e-beam provides better control over the e-beam and results in smaller diameter of the e-beam. Dwelling time of the e-beam 420 over the portion of the defect 407 depends on the height of the portion of the defect 407 and may be used to control a depth of etching. For one embodiment, current supplied to a source of the e-beam 420 to etch the defect 403 of quartz having a size in the approximate range of 20 nm to 100 nm is in the approximate range of 15 picoampers (“pA”) to 40 pA, and dwelling time may be in the approximate range of 1 μsec to 10 μsec. For one embodiment, repair boxes may be used to correlate the dwelling time with the size of the defect. Each of the repair boxes has dimensions that correspond to a size of a portion of the defect 407, wherein the size may be derived from a three-dimensional profile (“map”) of the defect 407 produced by one of the methods described above. For one embodiment, different repair boxes are generated for different types of the defects on the APS mask 400. Generation of repair boxes to define a size of an object is a technique, which is known to one of ordinary skill in the art of microscopic image mapping. For an embodiment, to perform e-beam induced etching of the defect 407 on the plate 402 of the APS mask 400 any Scanning Electron Microscopy (“SEM”) based e-beam equipment, for example, a MeRiT™ MG (Trademark) e-beam system produced by NaWoTec-Carl Zeiss, located in Germany, may be used. Next, an absorbing layer (“absorber”) having an overhung structure is reconstructed on the plate 402 of the APS mask 400.

FIG. 4D is a side view 443 of the APS mask 400, wherein the e-beam 420 is used to reconstruct an absorber 423 having an overhung structure 425 on the plate 402 according to one embodiment of the invention. As shown in FIG. 4D, a precursor gas 422 is dispensed near the e-beam 420 over a surface 424 of the plate 402 adjacent to the absorber 401. Deposition of a material on the plate 402 is enabled by the e-beam 420 focused on the surface 424 of the plate 402, as shown in FIG. 4D. For one embodiment, the material deposited on the surface 424 of the plate 402 using the e-beam 420 may be any opaque material that is preserved during cleaning of the APS mask 400. For one embodiment, the material deposited on the surface 424 of the plate 402 using the e-beam 420 may be any material that is opaque to radiation, which is an X-ray, an extreme ultra violet (“EUV”) light, an ultra violet (“UV”) light, or any combination thereof. For another embodiment, the absorber 423 having the overhung structure 425 may be deposited on the surface 424 of the plate 402 using a focused ion beam (“FIB”).

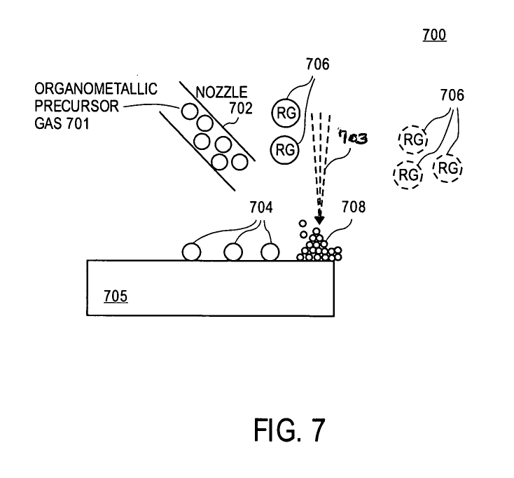

FIG. 7 illustrates a schematics 700 of depositing 700 of an absorbing layer having an overhung structure on a plate of an APS mask using an e-beam induced deposition according to one embodiment of the invention. A precursor gas 701 is introduced through a nozzle 702 near a focused e-beam 703. As shown in FIG. 7, molecules 704 of the precursor gas 701 introduced at a surface of a plate 705 are fragmented by primary electrons of the e-beam 703, which results in deposition the molecules and atoms of the material of the absorber layer 708 on the plate 705, and formation of residual gas 706.

Referring back to FIG. 4D, for one embodiment, to deposit the absorber 423, the precursor gas 422, which includes a metal may be used. For another embodiment, the precursor gas 422 includes carbons. For yet another embodiment, the precursor gas 422 includes a metal, carbons, for example, hydrocarbons, and any combination thereof. For an embodiment, the precursor gas 422 to deposit the absorber 423 includes Pt—CH, for example, methylcyclopentadienyl platinum (CH₃C₅H₄) Pt (CH₃)₃. The e-beam 420 induces dissociation of the precursor gas of Pt—CH into H₂, CHx fragments, and Pt-carbon compounds. As a result of the dissociation induced by the e-beam 420, hydrogen gas (“H₂”) and other volatile by-products go away from the surface 424, and Pt-carbon compounds and other non-volatile by-products, for example, other carbon-compounds deposit on the surface 424 of the plate 402. For another embodiment, the precursor gas 422 to deposit the absorber 423 includes tungsten carbonyls, for example, W(CO)₆, WF₆, methane, or any combination thereof. For an embodiment, the absorber 423 having the overhung structure 425 on the surface 424 is thick enough to completely block light. For an embodiment, a thickness of the absorber 423 having the overhung structure 425 on the surface 424 is in the approximate range of 100 nm to 500 nm. A voltage of the e-beam 420 is used to limit charging of the surface of the mask 402. For one embodiment, a voltage of the e-beam 420 to deposit absorber 423 is in the approximate range of 0.8 kilovolts (“kV”) to 1.5 kV, and more specifically, about 1 kV. For an embodiment, if the mask 402 has less charging, for example, a contact layer mask, voltage of the e-beam 420 may be at least 1 kV. Typically, higher voltage of the e-beam 420 provides higher spatial resolution. The e-beam 420 dwells above the surface 424 of the plate 402 for a predetermined time and then moves by a predetermined step, which is defined by a pixel spacing, to the next point above the surface 424 of the plate 402. The dwelling and moving of the e-beam 420 is continuously repeated until the absorber 423 having a predetermined thickness is formed on the surface 424. For one embodiment, the overhung structure 425 of the absorber 423 is not supported by the surface 424 of the plate 402. Dwelling and moving of the e-beam 420 may be performed using a raster scan technique, or a serpentine scan technique described above with respect to etching of the defect 407. For an embodiment, for the depositing of the absorber 423 having the overhung structure 425 the frame refresh time for the e-beam 420 is shorter relative to the frame refresh time for the etching. For an embodiment, the predetermined time for dwelling of the e-beam 420 is long enough, and the predetermined step to move the e-beam 420 from one point to the next point along the surface 424 is small enough to provide chemical bonding of the molecules deposited on the surface 424 to form the opaque material. More specifically, the predetermined time for dwelling of the e-beam 420 over one point of the surface 424 is in the approximate range of 1 μsec to 10 μsec, and the predetermined step to move the e-beam 420 from one point to the next point along the surface 424 is in the approximate range of 1 nm to 10 nm. For an embodiment, the APS mask 400 may be positioned relative to the incident e-beam at any angle to allow the absorber 423 be built at any angle and any orientation relative to the surface 424 of the APS mask 400. For one embodiment, the overhung structure 425 of the absorber 423 deposited on the surface 424 may be up to 1 μm long. More specifically, the length of the overhung structure 425 of the absorber 423 deposited on the surface 424 may be in the approximate range of 10 nm to 150 nm.

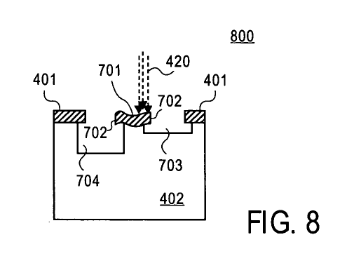

FIG. 8 is a side view 800 of the APS mask 400, wherein the e-beam 420 is used to reconstruct a missing absorber 701 having overhung structures 702 at both sides of trenches 703 and 704 in the plate 402 according to another embodiment of the invention.

The missing absorber 701 having the overhung structures 702 is reconstructed by using the e-beam 420. Reconstructing of the missing absorber 701 is performed by e-beam induced deposition of an opaque material having a predetermined thickness to block light using a process described above with respect to FIG. 4D.

For alternate embodiments, the methods described above may be used to repair various types of defects in masks. The defects include missing absorbers, superfluous absorbers, defects of a substrate (“plate”) of a mask at various locations of the mask, for example, under overhung, at the bottom of a comb of a plate, at the edge of a comb of a plate, or any combination thereof. For alternate embodiments, the methods described above may be used to repair masks for variety of applications, for example, Extreme Ultra Violet (“EUV”) masks, Electron Projection Lithography (“EPL”) masks, low energy EPL (“LEEPL”) masks, imprint lithography masks, or to any combination thereof.

What is claimed:

1. A method to repair a mask, comprising:
removing an absorbing layer over a defect on a plate;
removing the defect on the plate using an e-beam; and
reconstructing the absorbing layer having an overhung structure on the plate.

2. The method of claim 1, wherein removing the absorbing layer comprises cutting the absorbing layer down to the plate using a tip of an atomic force microscope.

3. The method of claim 1, wherein removing the absorbing layer comprises e-beam induced etching.

4. The method of claim 1, wherein removing the defect comprises:
cutting a first portion of the defect; and
etching a second portion of the defect with a first chemistry, wherein etching is induced by the e-beam.

5. The method of claim 4, wherein the defect on the plate comprises quartz and the first chemistry is formed using a gas, which includes fluorine.

6. The method of claim 1 further comprising:
generating a profile of the defect on the plate to control removing the defect on the plate.

7. The method of claim 1, wherein reconstructing the absorbing layer includes
depositing a material on the plate using a second chemistry, wherein depositing is induced by the e-beam.

8. The method of claim 7, wherein the second chemistry is formed using a gas, which includes metal carbohydrates.

9. The method of claim 7, wherein the material is opaque to a radiation, wherein the radiation is selected from a group consisting of an X-ray, an extreme UV light, an UV light, and any combination thereof.

10. The method of claim 1, wherein the overhung structure has a length in an approximate range of 20 nm to 150 nm.

11. The method of claim 1, wherein the absorbing layer has a thickness in the approximate range of 20 nm to 100 nm.

12. The method of claim 1, wherein the absorbing layer includes chrome.

13. The method of claim 1, wherein the absorbing layer includes tantalum nitride.

14. A method to repair a phase-shift mask, comprising:
removing an absorbing layer over a defect on a plate;
measuring a profile of the defect on the plate;
etching the defect on the plate using an e-beam utilizing the profile to control etching.

15. The method of claim 14 further comprising:
redepositing the absorbing layer having an overhung structure on the plate using the e-beam.

16. The method of claim 14, wherein removing the absorbing layer comprises:
cutting through the absorbing layer down to the plate using a tip of an atomic force microscope.

17. The method of claim 14, wherein removing the absorbing layer comprises e-beam induced etching.

18. The method of claim 14, wherein measuring the profile of the defect includes
measuring a height of the defect using the AFM tip; and
generating a repair box having dimensions that correspond to a size of a portion of the defect on the plate.

19. The method of claim 14, wherein etching the defect on the plate comprises:
dwelling the e-beam over the portion of the defect on the plate for a predetermined time defined by the profile of the defect; and
scanning the e-beam along the defect.

20. The method of claim 19, wherein scanning the e-bean includes performing a raster scan.

21. The method of claim 19, wherein scanning the e-beam includes performing a serpentine scan.

22. The method of claim 15 further comprising:
cleaning a surface of the plate before etching the defect using the e-beam, to remove one or more materials that include carbon.

23. A method to repair an alternating phase-shift mask, comprising:
mechanically removing an absorbing layer over a defect on the plate;
mechanically removing a first portion of the defect on the plate;
etching a second portion of the defect on the plate, wherein etching is induced by an e-beam; and
redepositing the absorbing layer having an overhung structure on the plate, wherein redepositing is induced by the e-beam.

24. The method of claim 23 further comprising:
removing a debris from a surface of the plate using a gas.

25. The method of claim 23 further comprising:
cleaning the surface of the plate to remove hydrocarbons prior to etching the second portion of the defect.

26. The method of claim 23, wherein etching the second portion of the defect includes
introducing a first gas over the second portion of the defect on the plate to form a first chemistry to etch the defect;
dwelling the e-beam over the second portion of the defect on the plate for a first predetermined time; and
moving the e-beam along the surface of the second portion of the defect by a first predetermined step to a next point over the surface of the second portion of the defect.

27. The method of claim 26, wherein dwelling the e-beam and moving the e-beam are continuously repeated until the second portion of the defect is removed.

28. The method of claim 26, wherein the first predetermined time for dwelling of the e-beam is sufficiently long for the first chemistry to perform etching of the second portion of the defect on the plate.

29. The method of claim 28, wherein the first predetermined time is from about 1 μsec to about 10 μsec.

30. The method of claim 26, wherein the second portion of the defect on the plate includes quartz and the first gas includes fluorine.

31. The method of claim 23, wherein redepositing the absorbing layer having the overhung structure includes
introducing a second gas over the plate to form a second chemistry;
dwelling the e-beam over the plate for a second predetermined time to induce forming the opaque material on the plate from a second chemistry of the second gas;
moving the e-beam by a second predetermined step.

32. The method of claim 31, wherein dwelling the e-beam and moving the e-beam are continuously repeated until the opaque material having the overhung structure on the plate is formed.

33. The method of claim 31, wherein the second gas includes organometallic compounds, hydrocarbons, carbonyls, fluorides, or any combination thereof.

34. The method of claim 31, wherein the second predetermined time for dwelling of the e-beam is sufficiently long and the second predetermined step to move the e-beam is sufficiently small to chemically bond molecules of the absorbing layer having the overhung structure.

35. The method of claim 34, wherein the second predetermined time for dwelling of the e-beam is from about 1 μsec to about 10 μsec and the second predetermined step is from about 1 nm to 10 nm.
